(19) United States
(12) Patent Application Publication
(10) Pub. No.: US 2011/0077650 A1
(43) Pub. Date: Mar. 31, 2011

(54) MEDICAL DEVICE AND METHOD

(75) Inventors: Armin Braun, Wermelskirchen (DE); Stefan Greuter, Tuttlingen (DE); Michael Lurz, Durbheim (DE)

(73) Assignee: SMITH & NEPHEW ORTHOPAEDICS GMBH, Tuttlingen (DE)

(21) Appl. No.: 12/866,700

(22) PCT Filed: Feb. 9, 2009
(86) PCT No.: PCT/EP2009/000884

§ 371 Date: Dec. 7, 2010

(30) Foreign Application Priority Data
Feb. 8, 2008 (GB) 0802402.8

(51) Int. Cl. A61B 17/62

(52) U.S. Cl. 606/56

(57) ABSTRACT

A device (1) for aligning a guide wire with a femur, comprising: an indication means (2) comprising at least two arms (3), each having a section (4) that is part-circular; an alignment means (6) for locating a portion of the bone for insertion of the guide wire, in use; and a body (18) connected to the indication means (2) and the alignment means (6), wherein the sections of the at least two arms (3) of the indication means (2) can be moved from an open to a closed position, and wherein in the open position the sections of the arms (3) can pass over the femoral head, and wherein in the closed position the sections of the arms (3) form a ring that has dimensions corresponding to a femoral implant component internal diameter. A system and method of aligning a guide wire with a femur.

The present invention relates to medical devices, in particular devices for aligning a guide wire (for example a k-wire) with respect to a bone. The present invention also relates to a method of aligning and inserting a guide wire into a bone.

Total hip replacements may fail prematurely due to excessive wear, particularly in active patients. Hence hip resurfacing, using metal on metal bearings, is increasingly being used with good results. Resurfacing preserves the patient's natural femoral neck and part of the femoral head. Accordingly, accurate positioning of the implant components is essential to preserve the integrity and strength of the natural bone. On the rare occasion that metal on metal resurfacings fail, it is mainly because of fracture of the femoral neck or loosening of the femoral component, which may result from poor surgical technique with notching of the femoral neck or incorrect angular positioning of the femoral component.

During the resurfacing operation, preparation of the femur starts with the positioning and drilling of a guide wire through the femoral head and into the neck. Guide wire position is critical because it will define the position and angle of the femoral component relative to the patient's femur. Clearly, it is best for the surgeon to position the guide wire correctly on the first attempt. Once the guide wire is inserted, its position may be verified by rotating a stylus around the femoral neck and the appropriate head component size is identified. The guide wire is then over drilled with a cannulated drill to increase the hole size. A guide rod is then inserted into the hole and used to guide a rotating cylindrical cutter to shape the femoral head into a cylinder. This is the stage in the operative procedure where notching of the femoral neck can occur due to incorrect positioning or over sailing of the cylinder cutter. A face cutter is then used to resect the unwanted bone. The guide rod is used to guide a rotating chamfer cutter to chamfer the proximal end of the cylinder. This procedure ensures that the implant component fits exactly to the bone.

The femoral head cannot be used as a positioning reference when placing the guide wire, because it is invariably misshapen in varying degrees due to the onset of arthritis. A preferred reference to use is the femoral neck, as this is where notching must be avoided, but this can also be partially misshapen due to osteophites.

Due to anxiety about notching the femoral neck and the smaller size of the neck relative to the femoral head, it is generally accepted that the best position for the guide wire and hence the femoral implant stem is in the exact centre of the femoral neck. This is often hard to determine because the neck cross section is not circular.

In addition to the guide wire being placed centrally in the neck, there are two important angles of the femoral implant axis relative to the femur which are described in different planes. Observed in the frontal (or coronal) plane on a frontal X-ray, varus/valgus angle is the angle between the shaft of the femur and the implant axis. The appropriate angle is somewhat patient specific, but generally within the range 135-145 degrees. The axis of the natural femoral neck is more varus (or more horizontal) and is difficult to judge because it tapers outwards towards the shaft of the femur. It is therefore erroneous to reference the natural neck angle as the appropriate angle for the implant axis. Excessive varus positioning of the implant is considered to be the second most contributory factor (after notching) towards femoral neck fracture and femoral component loosening.

Observed in the horizontal (or transverse) plane, version angle is a forward or backward angulation of the implant axis relative to the shaft of the femur. It is generally not apparent on X-ray but can be judged intra-operatively by observing the underside of the femoral neck. The appropriate angle is also patient specific but generally within the range 15-25 degrees. In this case, the surgeon generally tries to align the implant axis with the patient's natural anteversion angle.

It is generally accepted that a resurfacing head implanted with the appropriate varus/valgus and version/anteversion angles without notching of the femoral neck will have a good chance of success. However this goal is becoming more difficult to achieve, especially due to the limitations of minimally invasive surgery. There is an increasing trend towards minimally invasive surgery in hip resurfacing which reduces the amount of exposure, access and visibility to the femoral head and neck. It is more difficult for surgeons to detect and correct errors using their judgment, with reduced access and visibility. Therefore they are dependent on the effectiveness of the surgical instrumentation.

A number of devices exist to facilitate positioning of the guide wire and hence the femoral implant component. Early devices used a pin in the lateral femur to help determine angular position and a probe rotating around the neck to avoid notching. The requirement for a pin in the lateral femur means that such devices are not suitable for minimally invasive surgery because there is insufficient access to insert a pin laterally.

Later devices follow the trend towards minimally invasive surgery. The devices tend to fall into three categories, namely adjustable platform type, clamp type, and ring type devices.

Adjustable platform type devices comprise a drill guide and a platform that is fixed to the femoral head and from which adjustments to position and angles are made and verified with a rotating stylus. Such devices provide a stable platform to work from, but have the disadvantage that the surgeon still has to judge and fix varus/valgus and version angles simultaneously.

Clamp type devices comprise a drill guide and opposing jaws that attach to the femoral neck. A common problem with clamp type devices is that they tend to follow the natural femoral neck angle, which, as already described, is not the correct angle for the femoral implant axis. An attempt to overcome this has been made by replacing a symmetrical jaw clamp with an offset jaw clamp. Offsetting the jaws allows the device to be placed in a more valgus angle relative to the natural neck. However an offset jaw clamp is inherently unstable because the jaws do not directly oppose one another. It is therefore less effective as a clamp.

In both the above types of devices, it is a difficult task for the surgeon to decide varus/valgus and version angles simultaneously, particularly considering that these angles are judged in two different anatomical planes.

Known ring type devices comprise a drill guide and a partial or complete ring which is placed around the femoral neck, where the diameter of the ring corresponds to the femoral implant component internal diameter. In addition, existing ring type devices require the surgeon to manipulate the device from the side of the femoral neck, which means that the surgical operation is not minimally invasive. In addition, these devices are not as stable as clamp type devices because they do not attach to the femoral neck. Furthermore, varus/valgus and version angles must also be judged and fixed simultaneously by the surgeon when using such devices. Consequently, they present similar problems to those encountered with clamp type devices.

Accordingly, the present invention aims to maximise the accuracy of guide wire placement which in turn optimises the positioning of the final femoral component. The present invention also aims to provide guide wire placement devices that are suitable for use in minimally invasive surgery.

According to a first aspect of the present invention, there is provided a device for aligning a guide wire with a femur, comprising:
an indication means comprising at least two arms, each having a section that is part-circular;
an alignment means for locating a portion of the bone for insertion of the guide wire, in use; and
a body connected to the indication means and the alignment means,
wherein the sections of the at least two arms of the indication means can be moved from an open to a closed position, and wherein in the open position the sections of the arms can pass over the femoral head, and wherein in the closed position the sections of the arms form a ring that has dimensions corresponding to a femoral implant component internal diameter.

An advantage of the present invention is that it increases the accuracy of guide wire placement. Consequently, positioning of the final femoral component is optimised, significantly reducing, if not eliminating, failure of the metal on metal resurfacing. In addition, it does not require a lateral or posterior targeting pin and therefore is suitable for minimally invasive surgery since it can be operated through a reduced incision.

Another advantage of the present invention is that the sections of the arms of the indication means can pass over the femoral head from the top down, thereby enabling minimally invasive surgery. In contrast, known devices do not enable such top down access. Known devices require access from the femoral neck and/or require the surgeon to manipulate the device from the side of the femoral neck. Accordingly, such known devices require a significantly larger incision making them unsuitable for minimally invasive surgery.

Another advantage of the present invention is that the indication means provides the surgeon with a visual indication of whether a particular femoral implant, and hence corresponding sleeve cutter, is suitable or optimal for the operation.

In the closed position, the sections of the arms form a ring that may have dimensions corresponding to femoral implant component internal diameters in the range 25 mm to 80 mm. The corresponding internal diameter may be in the range 30 to 70 mm. The internal diameter may be in the range 30 to 60 mm. The internal diameter may be in the range 30 to 50 mm. The internal diameter may be in the range 30 to 45 mm. The internal diameter may be in the range 35 to 45 mm.

The proximal ends of the arms of the indication means may be connected to the body via a mechanism that enables movement of the arms between a closed and an open position.

According to preferred embodiments of the present invention, the mechanism is configured such that it enables the user to operate the device with one hand.

The mechanism may be a gear and lever-gear rack assembly. The mechanism may comprise a locking nut to lock the assembly in a particular position. The locking nut may be fully open, partially open, or locked. This has the advantage that the locking nut can act as a brake when partially open, enabling fine adjustment.

The mechanism may be a self-locking mechanism. The self-locking mechanism may be a ratchet mechanism having a release means for unlocking the arms.

The mechanism may comprise a resilient means for biasing the arms apart. The resilient means may be a spring.

The indication means may have two, three, four, five etc. arms. Preferably, the indication means has two arms.

According to preferred embodiments of the present invention, the sections of the two arms are half-circular. In such embodiments, the sections form a complete ring when closed. Compared to existing devices that only have a partial ring, this has the advantage that the surgeon can visualise the dimensions of the eventual implant at all points around the femoral neck.

The sections of the indication means may be reversibly connected to the at least two arms. The sections of the indication means may be mechanically attached to the at least two arms. The sections of the indication means may be magnetically attached to the at least two arms.

The indication means may be reversibly connected to the body.

The alignment means may be reversibly connected to the body.

The alignment means may receive a guide wire, in use.

The alignment means may comprise a drill guide.

Devices according to embodiments of the present invention may further comprise a fixation means. The fixation means may comprise at least one retractable spike. The fixation means may comprise a retractable spiked tube.

The fixation means may comprise a reamer dome shaped drill guide. This enables the surgeon to visualise the dimensions of the femoral reamer relative to the femoral head.

The fixation means may be part of the alignment means. The fixation means may comprise a drill guide.

In use, the fixation means engages with the femoral head, thereby improving the stability of the device.

According to some embodiments of the present invention, the device further comprises a second alignment means connected to the body.

The second alignment means may be reversibly connected to the body.

The second alignment means may be reversibly connected to different parts of the body. The second alignment means may be reversibly connected to a plurality of connection points on the body. The second alignment means may be reversibly connected to two, three, four, five etc: connection points on the body.

The second alignment means may be reversibly connected to four connection points on the body, each connection point being located at 90 degrees with respect to each other.

The second alignment means may receive a guide wire, in use.

The second alignment means may comprise a drill guide.

An advantage of having a second alignment means is that it enables a surgeon to position a guide wire in a range of positions. The surgeon can manipulate the main body of the device (for example, by rotating it about its main axis and/or by tilting it) and independently manipulate the second alignment means, giving the surgeon a full angular range to work with. This enables the surgeon to visually compare the set-up planes of the femoral head/neck with those of the device. Accordingly, the present invention overcomes the prior art problem of the surgeon having to judge varus/valgus and version angles simultaneously.

The device may further comprise a resilient means that biases the sections of the arms of the indication means in a proximal direction. In use, the sections of the arms engage with the femoral head/neck junction. The sections are therefore positioned in the optimal position for determining the correct implant size. Such engagement with the femoral head/neck junction also stabilises the device and holds it in position on the femur.

The resilient means may be disposed in the first alignment means. For example, the alignment means may comprise a fixation means in the form of a retractable spiked tube that is spring-loaded.

The device may further comprise at least one fiducial for use in Computer Assisted Surgery (CAS). The at least one fiducial may be reversibly attached to suitable parts of the device.

The device may be made of any material suitable for a surgical environment. The device may be made of metal. The metal may be stainless steel. The metal may be titanium. The device may be made of a metal alloy.

The device may be made of a plastics material. The device may be made of a high density polymer.

The device may be made of a combination of materials selected from the group consisting of metal, metal alloy and plastics materials.

According to a second aspect of the present invention, there is provided a system comprising a device according to the first aspect of the present invention and a plurality of sections having dimensions corresponding to a range of femoral implant component internal diameters.

According to a third aspect of the present invention, there is provided a method of aligning a guide wire with a femur, comprising the steps of:
providing a device according to the first aspect of the present invention and a power source;
opening the sections of the at least two arms of the indication means such that the sections of the arms can pass over the femoral head;
passing the sections of the arms over the femoral head;
closing the sections of the arms such that they form a ring indicating the internal diameter of a femoral implant component;
moving the device so that the alignment means locates a portion of the bone for insertion of the guide wire; and
inserting the guide wire into the bone using the power source.

According to a fourth aspect of the present invention, there is provided a method of aligning a guide wire with a femur, comprising the steps of:
providing a system according to the second aspect of the present invention and a power source;
selecting at least two sections from the plurality of sections having dimensions corresponding to a desired femoral implant component internal diameter;
attaching the at least two sections to the at least two arms;
opening the sections of the at least two arms of the indication means such that the sections of the arms can pass over the femoral head;
passing the sections of the arms over the femoral head;
closing the sections of the arms such that they form a ring indicating the internal diameter of a femoral implant component;
moving the device so that the alignment means locates a portion of the bone for insertion of the guide wire; and
inserting the guide wire into the bone using the power source.

The power source may be a rotary power source. The power source may be a drill.

Reference will now be made, by way of example, to the accompanying drawings, in which:

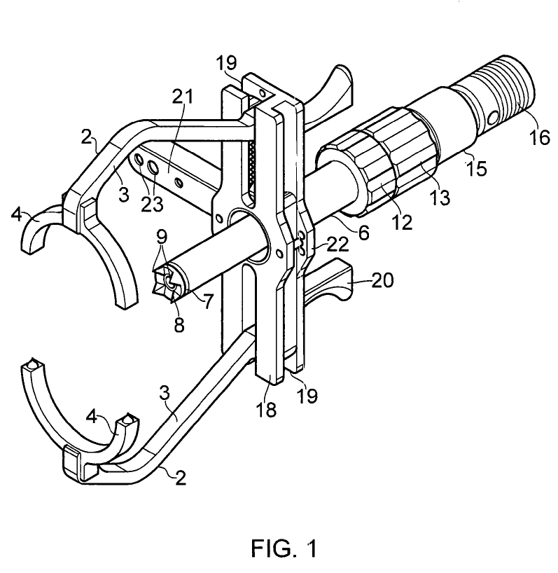

FIG. 1 is an isometric view of a device according to an embodiment of the present invention;

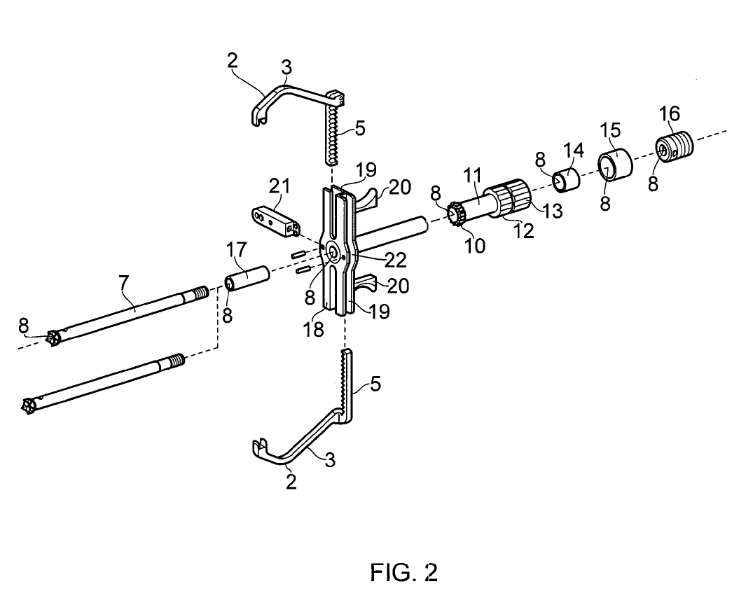

FIG. 2 is an exploded view of the device shown in FIG. 1; and

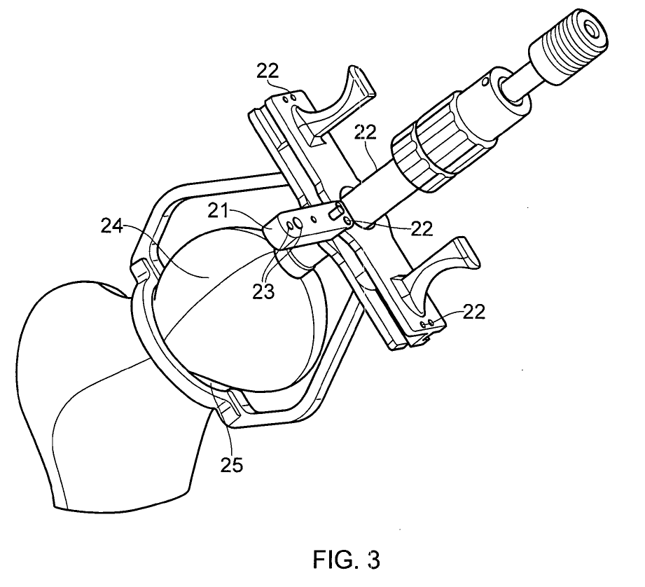

FIG. 3 is a side profile view of the device shown in FIG. 1 in use on a femur.

As shown in FIGS. 1 and 2, the device 1 comprises an indication means 2 comprising two arms 3, each having a section 4 that is part-circular. In the embodiment shown, the sections 4 are in the form of half rings. Each arm 3 is attached to a respective lever-gear rack 5 (see FIG. 2), which will be described in more detail below.

The device 1 also comprises an alignment means 6 for locating a portion of the bone for insertion of the guide wire (not shown), in use. The alignment means 6 comprises a fixation means 7 in the form of a retractable spiked tube 7. Tube 7 has a conduit 8 for receiving a guide wire (not shown), in use. Tube 7 has a plurality of spikes 9 (four spikes in the embodiment shown) for engaging with the femoral head in order to provide stability when in use. The alignment means 6 also comprises a gear wheel 10 which engages, in use, with the lever-gear racks 5 (see FIG. 2). Shaft 11 connects gear wheel 10 to knob 12. A user rotates knob 12 and hence gear wheel 10, which in turn drives lever-gear racks 5. As can be appreciated from FIGS. 1 and 2, rotation of knob 12 in a clockwise direction causes arms 3 to move together until the half rings 4 form a complete ring. Rotation of knob 12 in an anti-clockwise direction causes arms 3 to move apart. A locking nut 13 disposed adjacent knob 12 locks the arms 3 in a chosen position.

The alignment means 6 comprises a resilient means in the form of a spring 14, which is held in place by locking nut 15 and knob 16. Spring 14 biases the locking nut 13 in the distal direction so that it engages knob 12.

The alignment means 6 comprises a spring 17 that biases the retractable spiked tube 7 in a distal direction, such that in use sections 4 of arms 3 are pulled against the underside of the femoral head (see FIG. 3), thereby stabilising the device on the femoral head.

Conduit 8 extends the full length of the alignment means 6, as shown in FIGS. 2 and 3. In use, the surgeon inserts a guide wire (not shown) into the conduit 8 via the opening in knob 16.

The indication means 2 and the alignment means 6 are connected to a body 18. Body 18 comprises two channels 19 that receive respective lever-gear racks 5. The lever-gear racks 5 slide within the channels 19 when driven by gear wheel 10. Body 18 also comprises two finger grips 20 that enable the surgeon to manipulate the device 1.

Device 1 comprises a second alignment means 21 that is reversibly connectable to different parts of the body 18. As shown in FIG. 3, alignment means 21 can be reversibly connected to four connection points 22 on the body 18, each connection point being located at 90 degrees with respect to each other. The second alignment means 21 has at least one conduit 23 for receiving a guide wire (not shown), in use. In the embodiment shown in FIGS. 1 to 3, the second alignment means 21 has two conduits 23. The conduits have different diameters corresponding to guide wires having different diameters. For example, the conduits may receive 2.4 mm or 3.2 mm k-wire.

When not in use, with the retractable spiked tube 7 fully retracted, the distance between the proximal end of knob 16 and the distal end of arms 3 may be in the range 140-200 mm. The distance may be in the range 140-180 mm. The distance may be in the range 150-180 mm. The distance may be in the range 150-170 mm.

Body 18 may have a length in the range 60-120 mm. The length may be in the range 60-100 mm. The length may be in the range 70-100 mm. The length may be in the range 70-90 mm.

In the embodiment shown in FIGS. 1 to 3, each arm 3 has a flattened v-shape, with the apex disposed nearer to the distal end of the arm than the proximal end. The distance measured in a straight line between the proximal and distal ends of each arm 3 may be in the range 40-100 mm. The distance may be in the range 40-90 mm. The distance may be in the range 40-80 mm. The distance may be in the range 50-80 mm. The distance may be in the range 50-70 mm.

When the arms 3 are in their fully open position, the distance measured between the apex of each arm may be in the range 70-140 mm. The distance may be in the range 80-130 mm. The distance may be in the range 90-120 mm. The distance may be in the range 100-120 mm.

When the sections 4 of arms 3 are in their fully closed position, the internal diameter of the ring formed by the sections may be in the range 25 to 80 mm.

The device 1 is operated as follows. The device 1 may be held in one hand at the body 18 over the femoral head 24. The correctly sized set of sections 4 is attached to the arms 3. By turning the knob 12 anti-clockwise the arms 3 move outwards enlarging the opening between the sections 4. The retractable spiked tube 7 is then positioned on top of the femoral head 24 approximately centrically. The body 18 is pressed towards the femoral head 24 and by turning knob 12 in a clockwise direction the arms 3 move inwards towards the central axis until they eventually form a complete ring surrounding the femoral neck 25. Fixing the position of the arms 3 and sections 4 is done by turning the locking nut 13 clockwise. The surgeon can now release the pressure on the body 18. The spring loaded retractable spiked tube 7 maintains the position of the device 1 on the femur. The inner diameter formed by the sections 4 corresponds to the final diameter of the reamed femoral head 24 thus demonstrating the exit position of the femoral reamer. The outer diameter of the sections 4 is identical to that of the particular femoral implant selected. By retracting knob 16 the spring loaded retractable spiked tube 7 is released and can be freely positioned on the femoral head 24 to achieve the ideal axis and entrance point for the guide wire (k-wire). The surgeon can rotate the device 1 about the central axis of retractable spiked tube 7. The surgeon can also tilt the device 1.

To aid determination of the optimal position, in addition to positioning the main body of the device as described above, an additional alignment means 21 may be attached at 4 sides of the body 18 disposed at 90° angles. This enables the surgeon to visually compare the set-up planes of the femoral head 24/neck 25 with that of the device 1. Once the final position of the device 1 is determined a guide wire can be placed through the conduit 8 in retractable spiked tube 7.

To remove the device 1 the body 18 is again pressed towards the femoral head 24 and the locking nut 13 is turned anti-clockwise to release knob 12 and open the arms 3 and sections 4. The device can then be removed from the femoral head 24.

Due to the mechanism for moving the arms 3 being controlled by a knob 12 that is coaxial with the alignment means, the surgeon can operate the device with minimal access and therefore significantly reduce trauma.

Accordingly, another advantage of the device shown in FIGS. 1 to 3 is that the device can be used with one hand, providing the surgeon with a free hand during the procedure.

What is claimed is:

1. A device for aligning a guide wire with a femur, comprising: an indication means comprising at least two arms, each having a section that is part-circular; an alignment means for locating a portion of the bone for insertion of the guide wire, in use; and a body connected to the indication means and the alignment means, wherein the sections of the at least two arms of the indication means can be moved from an open to a closed position, and wherein in the open position the sections of the arms can pass over the femoral head, and wherein in the closed position the sections of the arms form a ring that has dimensions corresponding to a femoral implant component internal diameter.

2. A device according to claim 1, wherein the proximal ends of the arms of the indication means are connected to the body via a mechanism that enables movement of the arms between a closed and an open position.

3. A device according to claim 2, wherein the mechanism is configured such that it enables the user to operate the device with one hand.

4. A device according to claim 2, wherein the mechanism may be a gear and lever-gear rack assembly.

5. A device according to claim 2, wherein the mechanism comprises a lock for locking the indication means in a particular position.

6. A device according to claim 2, wherein the mechanism comprises a resilient means for biasing the arms apart.

7. A device according to claim 6, wherein the resilient means is a spring.

8. A device according to claim 1, wherein the sections of the indication means are reversibly connected to the at least two arms.

9. A device according to claim 8, wherein the sections of the indication means are magnetically attached to the at least two arms.

10. A device according to claim 1, wherein the indication means comprises two arms.

11. A device according to claim 10, wherein the sections of the two arms are half-circular.

12. A device according to claim 1, wherein the indication means is reversibly connected to the body.

13. A device according to claim 1, wherein the alignment means is reversibly connected to the body.

14. A device according to claim 1, wherein the alignment means receives a guide wire, in use.

15. A device according to claim 1, wherein the alignment means comprises a drill guide.

16. A devices according to claim 1, comprising a fixation means.

17. A device according to claim 16, wherein the fixation means is part of the alignment means.

18. A device according to claim 16, wherein the fixation means comprises a retractable spiked tube.

19. A device according to claim 16, wherein the fixation means comprises a drill guide.

20. A device according to claim 19, wherein the fixation means comprises a reamer dome shaped drill guide.

21. A device according to claim 1, comprising a second alignment means connected to the body.

22. A device according to claim 21, wherein the second alignment means is reversibly connected to the body.

23. A device according to claim 22, wherein the second alignment means is reversibly connected to four connection points on the body, each connection point being disposed at 90 degrees with respect to each other.

24. A device according to claim 21, wherein the second alignment means receives a guide wire, in use.

25. A device according to claim 1, comprising a resilient means that biases the sections of the arms of the indication means in a proximal direction.

26. A device according to claim 25, wherein the resilient means is disposed in the first alignment means.

27. A device according to claim 26 when dependent on claim 18, wherein the resilient means is disposed in the retractable spiked tube.

28. A device according to claim 25, wherein the resilient means is a spring.

29. A device according to claim 1, comprising at least one fiducial for use in computer assisted surgery.

30. A device according to claim 29, wherein the at least one fiducial is reversibly attached to the device.

31. A system comprising a device according to claim 1 to 30 and a plurality of sections having dimensions corresponding to a range of femoral implant component internal diameters.

32. A method of aligning a guide wire with a femur, comprising the steps of: providing a device according to claim 1 to 30 and a power source; opening the sections of the at least two arms of the indication means such that the sections of the arms can pass over the femoral head; passing the sections of the arms over the femoral head; closing the sections of the arms such that they form a ring indicating the internal diameter of a femoral implant component; moving the device so that the alignment means locates a portion of the bone for insertion of the guide wire; and inserting the guide wire into the bone using the power source.

33. A method of aligning a guide wire with a femur, comprising the steps of: providing a system according to claim 31 and a power source; selecting at least two sections from the plurality of sections having dimensions corresponding to a desired femoral implant component internal diameter; attaching the at least two sections to the at least two arms; opening the sections of the at least two arms of the indication means such that the sections of the arms can pass over the femoral head; passing the sections of the arms over the femoral head; closing the sections of the arms such that they form a ring indicating the internal diameter of a femoral implant component; moving the device so that the alignment means locates a portion of the bone for insertion of the guide wire; and inserting the guide wire into the bone using the power source.

34. A method according to claim 32, wherein the power source is a rotary power source.

35. A method according to claim 34, wherein the power source is a drill.

36. (canceled)

37. (canceled)

38. (canceled)
